(19) United States
(12) Patent Application Publication
(10) Pub. No.: US 2025/0359873 A1
(43) Pub. Date: Nov. 27, 2025

(54) INTRAOSSEOUS ACCESS ASSEMBLY

(71) Applicant: North American Rescue, LLC, Greer, SC (US)

(72) Inventors: Scotty Bolleter, San Antonio, TX (US); Brent Bronson, Duncan, SC (US); Jennifer Achay, Seguin, TX (US); Ethan Miles, Columbus, GA (US)

(21) Appl. No.: 18/672,278

(22) Filed: May 23, 2024

(51) Int. Cl. A61B 17/16; A61B 17/00; A61M 25/00; A61M 25/01

(52) U.S. Cl. CPC A61B 17/164; A61B 2017/00292; A61M 25/0082; A61M 25/0097; A61M 25/0102

(57) ABSTRACT

An intraosseous (IO) access assembly includes a driver defined between a proximal driver end (PDE) and a distal driver end (DDE). The driver defines a gripping portion between the PDE and the DDE. A stylet extends from the DDE of the driver. The stylet extends between a proximal stylet end (PSE) and a distal stylet end (DSE). A catheter hub is removably couplable to the driver at the DDE. A catheter extends from the catheter hub. The catheter extends between a proximal catheter end (PCE) and a distal catheter end (DCE). The catheter defines a longitudinal opening at the DCE. The stylet extending from the DDE of the driver is selectively received through the catheter hub and the catheter. The stylet having a tip defined at the DSE and extending through the opening at the DCE. The intraosseous (IO) access assembly forming a cutting tip at the DCE and DSE.

### FIELD OF THE DISCLOSURE

The present subject matter relates generally to an intraosseous (IO) access device assembly with features for adult and pediatric osteo-insertion and intramedullary access such as by providers of emergency medicine (austere, humanitarian aid, prehospital/field, and hospital based), critical care, surgery, and other types of medicine (Hematology/Oncology) where access to, and use of, the intramedullary space and vascular connectivity is desired.

### BACKGROUND OF THE DISCLOSURE

In the field of medical and trauma care for the treatment of civilian and military illness and injury, there exists a dedicated focus on mitigating preventable causes of death and the relief of suffering. For example, scenarios exist where immediate, and/or specific intramedullary/vascular access to a patient is crucial, but traditional peripheral or central venous access may be inappropriate, difficult, or impossible to obtain. Accordingly, IO assemblies may be used to provide access for assessment, delivery of fluids, medications, and/or blood, or blood products. IO devices may be inserted via three well established methods: manually assisted insertion, impact (spring or hammer) assisted insertion, and electrically or mechanically drill assisted insertion. Challenges arise with each method of insertion including the time and dexterity it takes to insert a manually assisted IO insertion; the feel or experience needed for impact assisted IO insertion; or lastly, the prohibitively high cost or complexity of powered IO devices, as well as the availability of batteries, electricity, or experience needed to use them.

In order to minimize one or more of the above challenges, it would be advantageous to have an IO assembly that offers medical providers insertion options and improved handling characteristics. For example, in the civilian or military medical setting, having an IO assembly that could allow a provider to select a situationally and provider appropriate insertion method may be desirable.

### BRIEF DESCRIPTION OF THE DISCLOSURE

Aspects and advantages of the invention will be set forth in part in the following description, or may be obvious from the description, or may be learned through practice of the invention.

In one example embodiment, an intraosseous access assembly is provided. The intraosseous access assembly defines an axial direction, a radial direction, and a circumferential direction. The intraosseous access assembly includes a driver defined between a proximal driver end (PDE) and a distal driver end (DDE). The driver defines an ergonomic gripping portion between the PDE and the DDE and a plurality of prongs extend from the DDE. The intraosseous access assembly also includes a stylet extending from the DDE of the driver. The stylet extends between a distal stylet end (DSE) and a proximal stylet end (PSE). The intraosseous access assembly further includes a catheter hub removably couplable to the driver at the DDE. The catheter hub defines a plurality of apertures for receiving respective prongs of the plurality of prongs of the driver. A catheter extends from the catheter hub. The catheter extends between a proximal catheter end (PCE) and a distal catheter end (DCE). The catheter defines a longitudinal opening at the DCE. The stylet extending from the DDE of the driver is selectively received through the catheter hub and the catheter. The stylet has a tip defined at the DSE and extends through the opening at the DCE.

In another example embodiment, an intraosseous access assembly is provided. The intraosseous access assembly defines an axial direction, a radial direction, and a circumferential direction. The intraosseous access assembly includes a driver defined between a proximal driver end (PDE) and a distal driver end (DDE) in the axial direction. The driver defines a gripping portion between the PDE and the DDE. The driver is configured to withstand impact along the axial direction. The driver includes a driving portion defined in the PDE. The driving portion defines a female receiving end to accept a complementary/matched male tool bit, enabling mated rotation of the driver. The intraosseous access assembly also includes a stylet extending from the DDE of the driver. The stylet extends between a distal stylet end (DSE) and a proximal stylet end (PSE). The intraosseous access assembly further includes a catheter hub removably couplable to the driver at the DDE. A catheter extends from the catheter hub. The catheter extends between a proximal catheter end (PCE) and a distal catheter end (DCE). The catheter defines a longitudinal opening at the DCE. The stylet extending from the DDE of the driver is selectively received through the catheter hub and the catheter. The stylet has a tip defined at the DSE and extends through the opening at the DCE.

In another example embodiment, an intraosseous access assembly is provided. The intraosseous access assembly defines an axial direction, a radial direction, and a circumferential direction. The intraosseous access assembly includes a driver defined between a proximal driver end (PDE) and a distal driver end (DDE). The driver defines a gripping portion between the PDE and the DDE and a plurality of prongs extend from the DDE. The intraosseous access assembly also includes a stylet extending between a distal stylet end (DSE) and a proximal stylet end (PSE). The intraosseous access assembly further includes a catheter hub removably couplable to the driver at the DDE. The catheter hub defines a plurality of apertures for receiving respective prongs of the plurality of prongs of the driver. A catheter extends from the catheter hub. The catheter extends between a proximal catheter end (PCE) and a distal catheter end (DCE). The catheter defines a cutting edge and a longitudinal opening at the DCE. The stylet extending from the DDE of the driver is selectively received through the catheter hub and the catheter. The stylet includes a tapered tip defined at the DSE and extends through the opening at the DCE. The cutting edge of the catheter and the tapered tip of the stylet form a cutting tip at the DSE.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides a perspective view of an intraosseous access assembly according to exemplary embodiments of the present disclosure.

FIG. 2 provides a cross-sectional side view of the exemplary intraosseous access assembly of FIG. 1.

FIG. 3 provides a perspective view of a driver of the exemplary intraosseous access assembly of FIG. 1.

FIG. 4 provides a side view of the driver of FIG. 2 according to exemplary embodiments of the present disclosure.

FIG. 5 provides a cross-sectional side view of the driver of FIG. 2 according to exemplary embodiments of the present disclosure.

FIG. 6 provides a front (distal) view of the driver of FIG. 3 according to exemplary embodiments of the present disclosure.

FIG. 7 provides a back (proximal) view of the driver of FIG. 3 according to exemplary embodiments of the present disclosure.

FIG. 8 provides a perspective view of a catheter hub of the exemplary intraosseous access assembly of FIG. 1.

FIG. 9 provides a cross-sectional side view of the catheter hub of FIG. 8 according to exemplary embodiments of the present disclosure.

FIG. 10 provides a front (proximal) view of the catheter hub of FIG. 8 according to exemplary embodiments of the present disclosure.

FIG. 11 provides a perspective (distal) view of a catheter tip according to exemplary embodiments of the present disclosure.

FIG. 12 provides a perspective (distal) view of a stylet tip according to exemplary embodiments of the present disclosure.

FIG. 13 provides a perspective (distal) view of the stylet tip of FIG. 12 within the (distal) catheter tip of FIG. 11 forming a cutting tip according to exemplary embodiments of the present disclosure.

Repeat use of reference characters in the present specification and drawings is intended to represent the same or analogous features or elements of the present invention.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the terms “first,” “second,” and “third” may be used interchangeably to distinguish one component from another and are not intended to signify location or importance of the individual components. The terms “includes” and “including” are intended to be inclusive in a manner similar to the term “comprising.” Similarly, the term “or” is generally intended to be inclusive (i.e., “A or B” is intended to mean “A or B or both”). In addition, here and throughout the specification and claims, range limitations may be combined or interchanged. Such ranges are identified and include all the sub-ranges contained therein unless context or language indicates otherwise. For example, all ranges disclosed herein are inclusive of the endpoints, and the endpoints are independently combinable with each other. The singular forms “a,” “an,” and “the” include plural references unless the context clearly dictates otherwise.

Approximating language, as used herein throughout the specification and claims, may be applied to modify any quantitative representation that could permissibly vary without resulting in a change in the basic function to which it is related. Accordingly, a value modified by a term or terms, such as “generally,” “about,” “approximately,” and “substantially,” are not to be limited to the precise value specified. In at least some instances, the approximating language may correspond to the precision of an instrument for measuring the value, or the precision of the methods or machines for constructing or manufacturing the components or systems. For example, the approximating language may refer to being within a 10 percent margin (i.e., including values within ten percent greater or less than the stated value). In this regard, for example, when used in the context of an angle or direction, such terms include within ten degrees greater or less than the stated angle or direction (e.g., “generally vertical” includes forming an angle of up to ten degrees in any direction, such as, clockwise or counterclockwise, with the vertical direction V).

The word “exemplary” is used herein to mean “serving as an example, instance, or illustration.” In addition, references to “an embodiment” or “one embodiment” does not necessarily refer to the same embodiment, although it may. Any implementation described herein as “exemplary” or “an embodiment” is not necessarily to be construed as preferred or advantageous over other implementations.

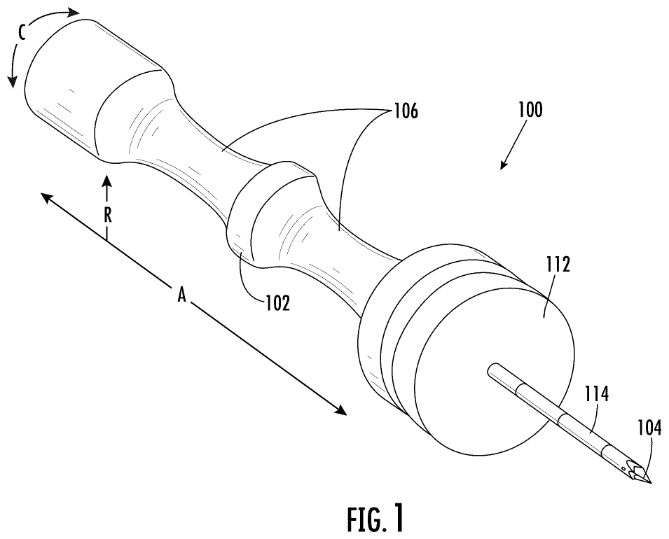

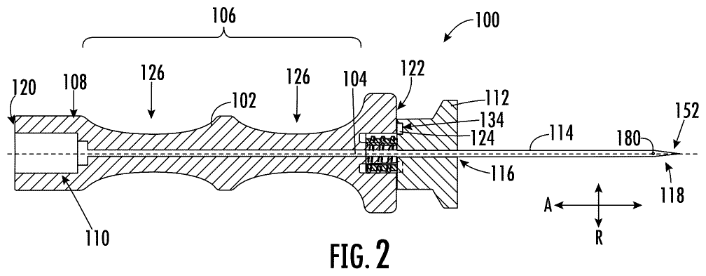

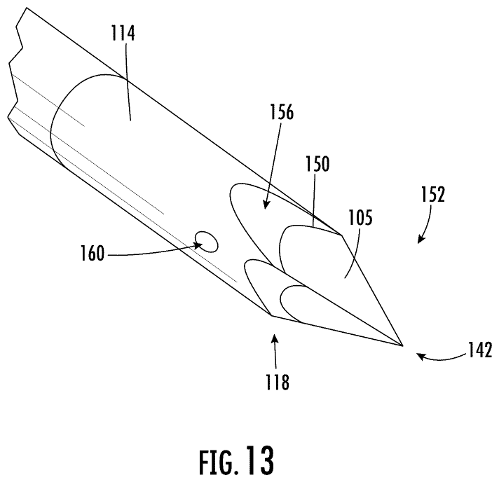

Referring now to the figures, FIGS. 1 through 2 provide a perspective view and a cross-sectional side view, respectively, of an assembled intraosseous access assembly 100 according to exemplary embodiments of the present disclosure. As shown, intraosseous access assembly 100 may define an axial direction A, a radial direction R, and a circumferential direction C. Intraosseous access assembly 100 may generally include a driver 102, a stylet 104 extending from driver 102, a catheter hub 112, and a catheter 114 extending from catheter hub 112. When driver 102 and stylet 104 are mated with catheter hub 112 and catheter 114, a cutting tip 152 (FIG. 13) of intraosseous access assembly 100 is formed for the periosteal and cancellous bone, as will be further described hereinbelow. In general, driver 102, and stylet 104 extending therefrom, may be separated/removed from catheter hub 112 and catheter 114. For example, assembled intraosseous access assembly 100 may generally place catheter 114 in a desired location, such as within an intramedullary space/bone of a patient requiring assessment and/or treatment, whereby driver 102, and stylet 104 extending therefrom, may be removed from catheter hub 112 and catheter 114 for the aspiration of blood, and/or the delivery of fluids, medications, blood, or blood products through catheter hub 112 and catheter 114, as will be described in further detail hereinbelow.

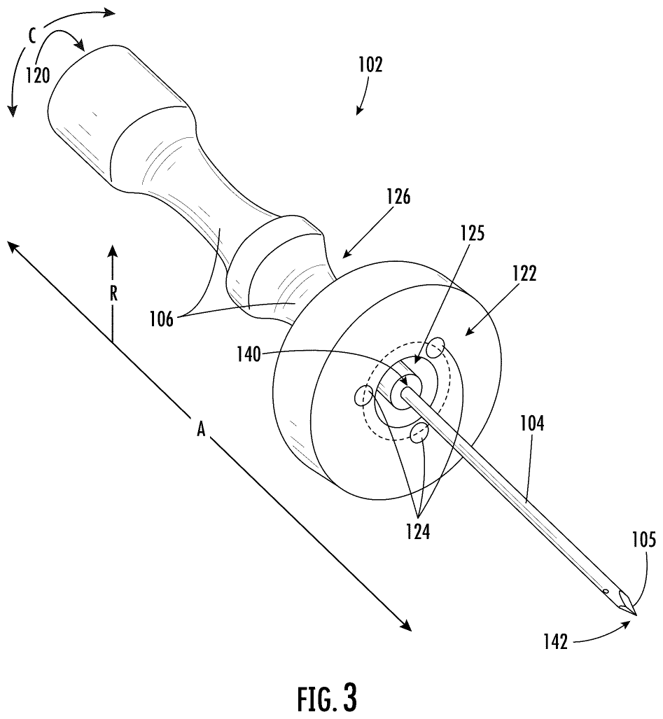

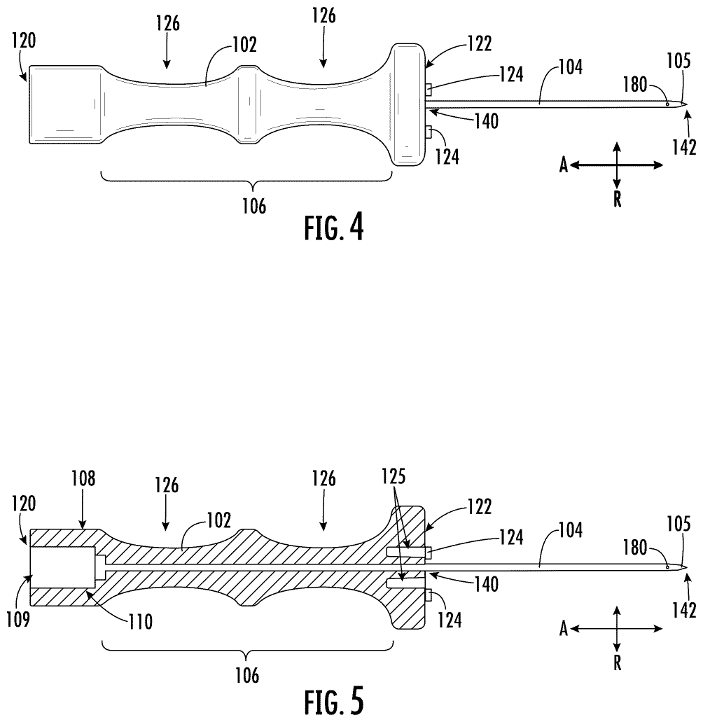

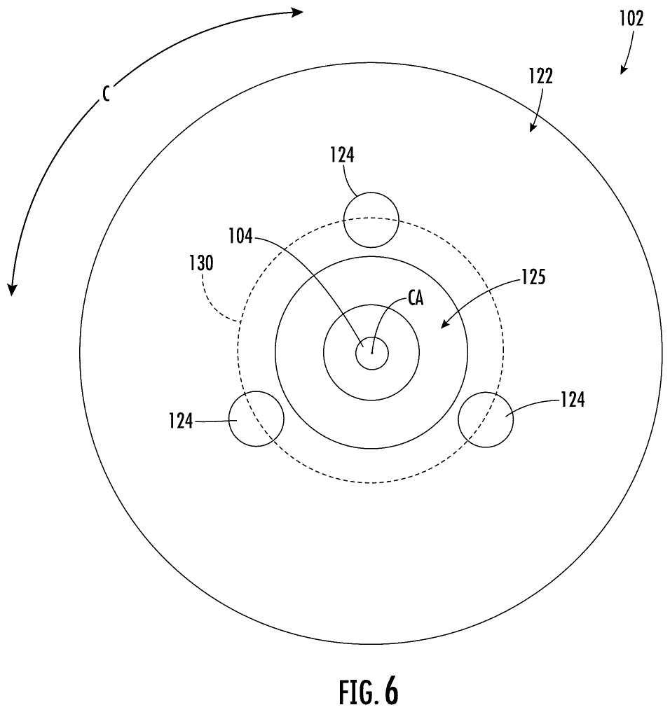

Referring now to FIGS. 3 through 7, provided are various views of driver 102 and stylet 104. In particular, FIG. 3 illustrates a perspective view of driver 102 of the exemplary intraosseous access assembly 100, FIG. 4 illustrates a side view of driver 102, FIG. 5 illustrates a cross-sectional side view of driver 102, and FIG. 6 illustrates a front (distal) view of driver 102. In general, as seen in FIG. 3, driver 102 may extend in the axial direction A between a proximal driver end (PDE) 120 and a distal driver end (DDE) 122. In general, stylet 104 may extend in the axial direction A from DDE 122, such as between a proximal stylet end (PSE) 140 and a distal stylet end (DSE) 142. Driver 102 may generally be cylindrically shaped down the axial direction of driver 102 with an ergonomic shape to facilitate and improve manual insertion. For example, driver 102 may extend radially, in the radial direction R, from a central axis CA (FIG. 2) around the circumferential direction C.

Referring to FIGS. 4 through 5, in the present exemplary embodiment, driver 102 may define an ergonomic gripping portion 106 between PDE 120 and DDE 122. Gripping portion 106 may generally include an hourglass shaped portion 126 between PDE 120 and DDE 122. For example, the present exemplary embodiment includes two (2) hourglass shaped portions 126 between PDE 120 and DDE 122, such as for a user to ergonomically position a thumb and fingers on/around gripping portion 106 for manual insertion of intraosseous access assembly 100. In particular, the present exemplary embodiment may include hourglass shaped portion 126 positioned proximate DDE 122 and distal to PDE 120. In general, hourglass shaped portion 126 may be radially narrower, e.g., in the radial direction R, than other portions of driver 102 along the axial direction A, such as a narrower portion may be axially bounded by three (3) wider portions, thus forming the hourglass shape. Some other example embodiments may include driver 102 with one (1) hourglass shaped portions 126, or more, such as three (3) or more hourglass shaped portions 126.

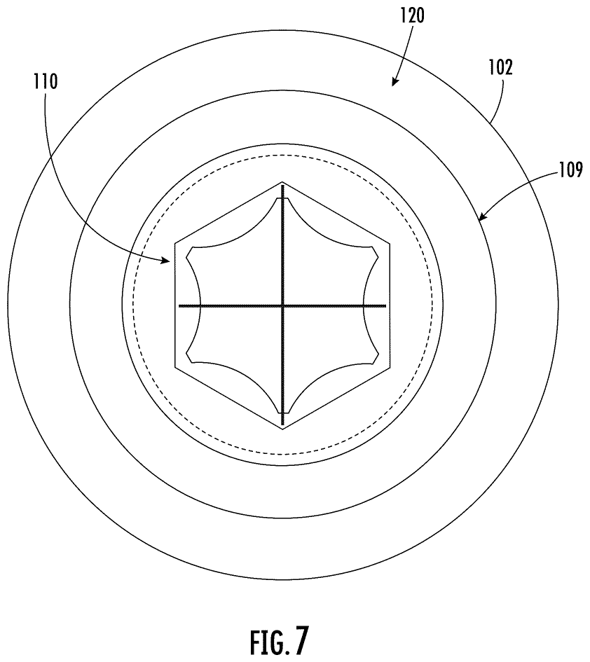

In general, driver 102 may configured to withstand impact along the axial direction A, such as by a hammer or any other suitable tool for impacting driver 102. Driver 102 may generally include an indentation, or, a driving portion 108 defined within PDE 120. Specifically, turning to FIG. 7, driving portion 108 may define a female receiving end 110, such as a modified female tool bit, uniquely configured to accept a host of complementary/matched male tool bits, such as standard “T” bit of select sizes, a standard Philips or flat bit of select sizes, or other like commercial driving bits. For example, mating a tool bit with female receiving end 110 may enable mated/powered rotation of driver 102 in either clockwise or counterclockwise direction about central axis CA. In some example embodiments, female receiving end 110 of driving portion 108 may include an opening 109 within driving portion 108 of driver 102. Accordingly, male tool bits may be accepted within opening 109 and engage female receiving end 110 for the mated/powered rotation of driver 102. As such, in some example scenarios, a power tool, such as a medical or commercially available drill, may be mechanically linked to driver 102 via driving portion 108 so as to drive powered rotation of driver 102 around central axis CA.

As may be seen in FIG. 6, driver 102 may define a plurality of prongs 124 extending outward, in the axial direction A, from DDE 122 and a slot 125 extending inward, in the axial direction A, from DDE 122. In particular, the plurality of prongs 124 may be spaced circumferentially, in the circumferential direction C, in an array 130 around central axis CA of driver 102. As shown, the plurality of prongs 124 may include three (3) prongs 124 spaced around array 130, however, other example embodiment may generally include one (1) or more prongs 124, such as four (4) prongs 124. Additionally, slot 125 may extend in the circumferential direction C around central axis CA of driver 102. In general, the plurality of prongs 124 may be magnetized to promote connectivity between driver 102 and catheter hub 112. The plurality of prongs 124 and slot 125 will be described further hereinbelow.

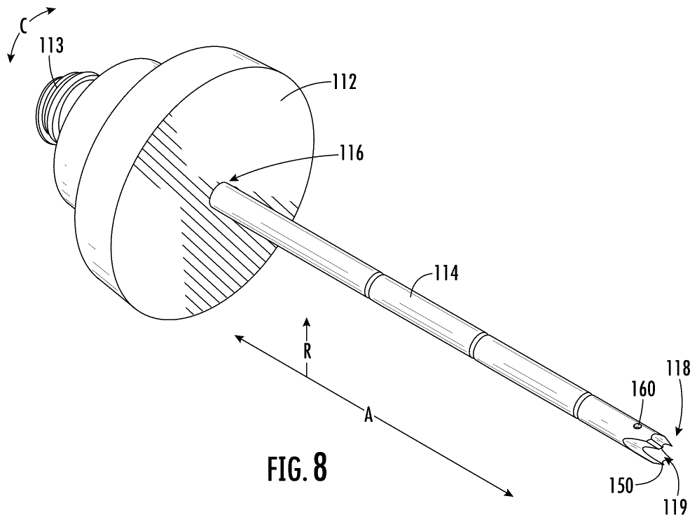

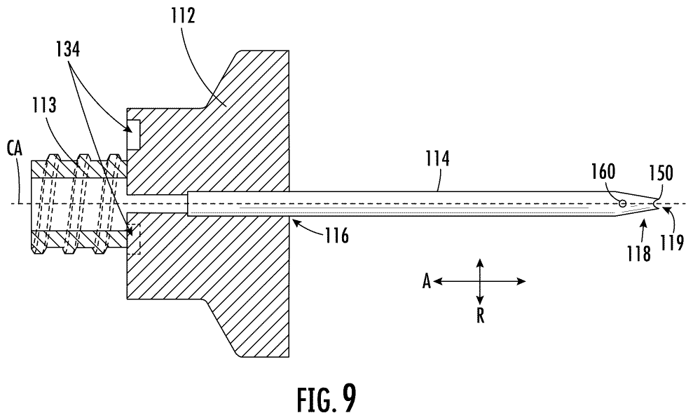

Turning now to FIGS. 8 through 10, provided are views of catheter hub 112 and catheter 114. In general, intraosseous access assembly 100 may generally include catheter hub 112, and catheter 114 extending from catheter hub 112. In general, FIGS. 8 through 9 illustrate catheter hub 112 including a threaded end 113, e.g., threaded end 113 may be standard medical Luer lock threads, generally configured to be accepted into slot 125 of driver (as seen in FIG. 2). Accordingly, slot 125 of driver 102 may accept but not interfere with threaded end 113 of catheter hub 112. Referring again to FIG. 9, the other side of catheter hub 112, i.e., opposite threaded end 113, may be catheter 114 extending in the axial direction A between a proximal catheter end (PCE) 116 and a distal catheter end (DCE) 118. In general, catheter 114 may include a longitudinal opening 119 at DCE 118 defined within a cutting (serrated) edge 150 of catheter 114. When combined with stylet 104 of driver 102, cutting edge 150 of catheter 114 may become a cutting tip as will be described in further detail hereinbelow.

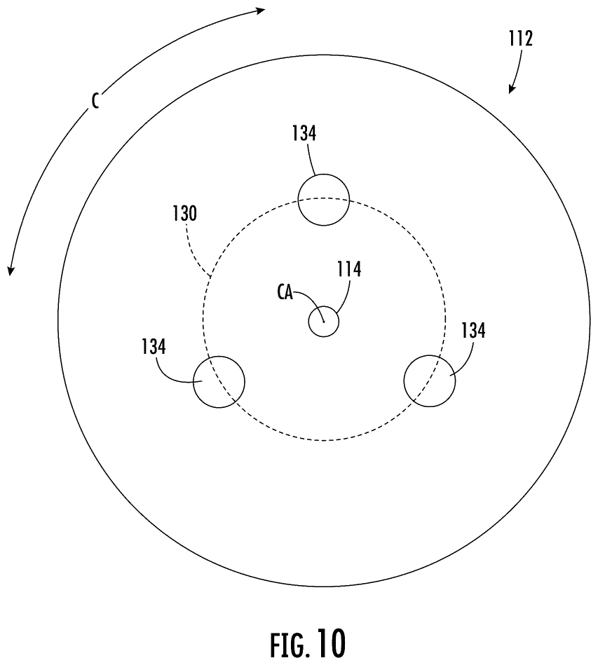

In general, catheter hub 112 may be removably couplable to driver 102 at DDE 122. As seen in FIGS. 9 through 10, catheter hub 112 may define a plurality of apertures 134 for receiving respective prongs of the plurality of prongs 124 of the driver 102. Accordingly, the plurality of apertures 134 may be spaced circumferentially, e.g., in the circumferential direction C, in array 130 around the axial direction A. As shown, the plurality of apertures 134 may include three (3) apertures 134 spaced around array 130, however, other example embodiment may generally include a complementary number of apertures 134 to prongs 124, such as one (1) or more apertures 134, such as four (4) apertures 134. In other words, the plurality of prongs 124 of driver 102 may be received in the plurality of apertures 134 of catheter hub 112 around a central axis CA of driver 102. Additionally, the plurality of apertures 134 may be magnetized complementary to the plurality of prongs 124. As such, the plurality of prongs 124 of driver 102 may be received in the plurality of apertures 134 of catheter hub 112, thereby locking and assuring, magnetically, connectivity to driver 102 and catheter hub 112 together with respect to rotation in either direction, circumferentially, around the central axis CA.

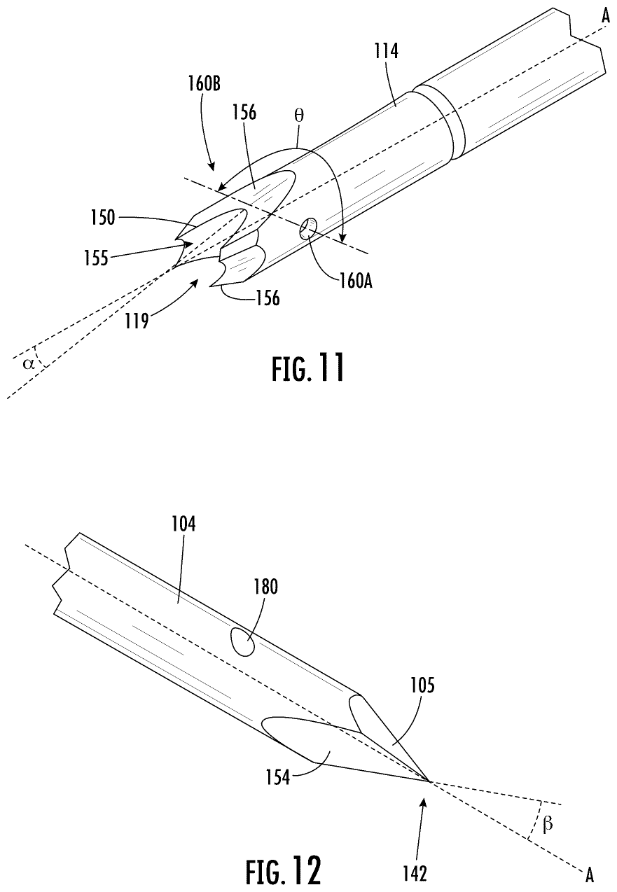

Looking now to FIGS. 11 through 12, illustrated are detailed perspective views of DCE 118 of catheter 114 (FIG. 11) and DSE 142 of stylet 104 (FIG. 12). In general, cutting edge 150 of catheter 114 may include a pair of tapers 156 defining a recessed portion 155 at the longitudinal opening 119 at DCE 118. In general, pair of tapers 156 may be on opposing sides, i.e., in the radial direction R. For instance, the pair of tapers 156 may be at a predetermined angle α. In some embodiments, α is about fifteen degrees (15°) from the axial direction A.

Separate from or in addition to the longitudinal opening 119, catheter 114 may define one or more lateral fenestrations (e.g., a plurality of fenestrations or openings 160A, 160B) in fluid communication (e.g., upstream fluid communication) with catheter 114. As shown, such lateral fenestrations 160A, 160B may be disposed rearward from the longitudinal opening 119 between the PCE 116 and the DCE 118. Nonetheless the lateral fenestrations 160A, 160B may each be disposed proximal to the DCE 118 (i.e., relatively distal, or further from the PCE 116). Moreover, the lateral fenestrations 160A, 160B may be longitudinally spaced apart (i.e., from each other) or disposed rearward from cutting edge 150 (i.e., such that cutting edge 150 is disposed at the DCE 118 and forward from the plurality of lateral fenestrations 160A, 160B. Notably, the lateral fenestrations 160A, 160B may allow fluid to be aspirated from or travel along catheter 114 even if the longitudinal opening 119 or one or more of the fenestrations 160A, 160B is/are obstructed.

In some embodiments, multiple lateral fenestrations 160A, 160B are circumferentially spaced apart (e.g., about a longitudinal direction or axis extending through the catheter 112 between the PCE 116 and the DCE 118). For instance, a predefined offset angle θ may be defined between circumferentially adjacent lateral fenestrations, e.g., 160A to 160B. Optionally, multiple adjacent pairs may be spaced apart by the same predefined offset angle θ.

In additional or alternative embodiments, multiple lateral fenestrations may be longitudinally spaced apart (e.g., along a longitudinal direction or axis extending through the catheter 114 between the PCE 116 and the DCE 118). For instance, a predefined longitudinal spacing may be defined between longitudinally adjacent lateral fenestrations. Optionally, multiple adjacent pairs may be spaced apart by the same longitudinal spacing (e.g., by the same distance or magnitude along the longitudinal direction).

Turning now to FIG. 12, stylet 104 may include a tapered tip 105 defined at DSE 142. In general, tapered tip 105 may include a plurality of tapers, such as first taper 154; first taper 154 may be at an angle β of between about ten to fifteen degrees (10° to 15°) from the axial direction A. Additionally, stylet 104 may define a hole 180 through stylet 104 proximate DSE 142, i.e., relatively distal or further from the PSE 140 than DSE 142. In particular, hole 180 may be configured to align with lateral fenestrations, e.g., 160A and 160B of catheter 114 when assembled.

Turning now to FIG. 13, intraosseous access assembly 100 may include stylet 104 extending from DDE 122 of driver 102 selectively received through catheter hub 112 and catheter 114. In particular, tapered tip 105 defined at DSE 142 of stylet 104 may extend through the longitudinal opening 119 at DCE 118 of catheter 114. As such, cutting edge 150 of catheter 114 and tapered tip 105 of stylet 104 may form a cutting tip 152 at the DCE and DSE 150 and 142. In other words, combining the pair of tapers 156 of cutting edge 150 of catheter 114 with the plurality of tapers of tapered tip 105 of stylet 104, may align and seamlessly form cutting tip 152 at DCE 150 and DSE 142 of the intraosseous access assembly 100. Notably, cutting tip 152 may be usable as a cutting utensil (e.g., in contrast to the pure puncture utensil provided by existing stylets) capable of penetrating surface portions of a patient's tissue (i.e., epidermis, dermis, and subcutaneous layers) and cutting into/through the patient's periosteum and cancellous bone. Advantageously, cutting tip 152 may be inserted without requiring a separate insertion utensil, as might be otherwise required in routine, difficult, or dynamic/austere settings.

Accordingly, in an example scenario, the intraosseous access assembly 100 may be used to provide immediate vascular access to a patient, such as for assessment or if the patient is in need of fluids, medications, blood, or blood products. A user may take the assembled intraosseous access assembly 100 and choose a desired insertion location, such the manubrium, body, proximal humerus, iliac crest, distal femur, proximal tibia, distal tibia, distal radius, or any other suitable bone as determined by those practiced in the art. In the present example scenario, the proximal tibia is chosen. The user may then choose an insertion method, e.g., manual, impact (hammer), or power assisted (drill) insertion. In the present example scenario, a drill may be available and is coupled to driving portion 108 of driver 102, whereby cutting tip 152 of the intraosseous access assembly 100 may be drilled into the proximal tibia. Once catheter 114 is set in the proximal tibia, driver 102, and stylet 104 extending therefrom, may be removed from within catheter hub 112 and catheter 114, such that catheter 114 and catheter hub 112 remain in position, providing intraosseous vascular access through catheter hub 112 and catheter 114 for the aspiration of blood or the delivery of fluids, medications, blood, or blood products to the patient.

As may be seen from the above, intraosseous access assembly 100 may be inserted via manual, impact, or power assisted insertion. Intraosseous access assembly 100 may include an hourglass shape to facilitate proper ergonomically enhanced handling and improve manual insertion. Additionally, intraosseous access assembly 100 may be configured to rotate either direction during manual or power assisted insertion via the plurality of prongs and the plurality of apertures mechanically linking the driver 102 to the catheter hub 112. Furthermore, cutting tip 152 of intraosseous access assembly 100 may advantageously enhance penetration through periosteum and cancellous bone. Additionally, the catheter may be fenestrated bilaterally to improve aspiration or flow and reduce the possibility of distal occlusion. Moreover, the catheter hub may include the Luer lock fitting such that standard, or typically available administration or extension sets, syringes and other medical devices may be fluidly coupled to the catheter hub.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. An intraosseous access assembly defining an axial direction, a radial direction, and a circumferential direction, the intraosseous access assembly comprising:
a driver defined between a proximal driver end (PDE) and a distal driver end (DDE), the driver defining an ergonomic gripping portion between the PDE and the DDE, the driver defining a plurality of prongs extending from the DDE;
a stylet extending from the DDE of the driver, the stylet extending between a distal stylet end (DSE) and a proximal stylet end (PSE);
a catheter hub removably couplable to the driver at the DDE, the catheter hub defining a plurality of apertures for receiving respective prongs of the plurality of prongs of the driver,
a catheter extending from the catheter hub, the catheter extending between a proximal catheter end (PCE) and a distal catheter end (DCE), the catheter defining a longitudinal opening at the DCE,
wherein the stylet extending from the DDE of the driver is selectively received through the catheter hub and the catheter, the stylet having a tip defined at the DSE and extending through the opening at the DCE.

2. The intraosseous access assembly of claim 1, wherein the plurality of prongs of the driver are received in the plurality of apertures of the catheter hub around a central axis of the driver, thereby locking the driver and the catheter hub with respect to rotation in either direction around the central axis.

3. The intraosseous access assembly of claim 2, wherein the plurality of prongs are spaced circumferentially in an array around the central axis of the driver.

4. The intraosseous access assembly of claim 1, wherein the gripping portion of the driver comprises an hourglass shaped portion between the PDE and the DDE.

5. The intraosseous access assembly of claim 1, wherein the catheter further defines a fenestration rearward from the longitudinal opening between the PCE and the DCE.

6. The intraosseous access assembly of claim 5, wherein the fenestration comprises a plurality of lateral fenestrations.

7. The intraosseous access assembly of claim 6, wherein each lateral fenestration of the plurality of lateral fenestrations is circumferentially spaced apart.

8. An intraosseous access assembly defining an axial direction, a radial direction, and a circumferential direction, the intraosseous access assembly comprising:
a driver defined between a proximal driver end (PDE) and a distal driver end (DDE) in the axial direction, the driver defining a gripping portion between the PDE and the DDE, the driver configured to withstand impact along the axial direction, the driver comprising a driving portion defined in the PDE, the driving portion defining a female receiving end to accept a complementary/matched male tool bit enabling mated rotation of the driver;
a stylet extending from the DDE of the driver, the stylet extending between a distal stylet end (DSE) and a proximal stylet end (PSE);
a catheter hub removably couplable to the driver at the DDE; and
a catheter extending from the catheter hub, the catheter extending between a proximal catheter end (PCE) and a distal catheter end (DCE), the catheter defining a longitudinal opening at the DCE,
wherein the stylet extending from the DDE of the driver is selectively received through the catheter hub and the catheter, the stylet having a tip defined at the DSE and extending through the opening at the DCE.

9. The intraosseous access assembly of claim 8, wherein the female receiving end of the driving portion defines an opening for receipt of the male tool bit within the PDE of the driver.

10. The intraosseous access assembly of claim 8, wherein the gripping portion of the driver comprises an hourglass shaped portion between the PDE and the DDE.

11. The intraosseous access assembly of claim 10, wherein the hourglass shaped portion of the gripping portion of the driver is positioned proximate the DDE.

12. The intraosseous access assembly of claim 8, wherein the catheter further defines a fenestration rearward from the longitudinal opening between the PCE and the DCE.

13. The intraosseous access assembly of claim 12, wherein the fenestration comprises a plurality of lateral fenestrations.

14. The intraosseous access assembly of claim 13, wherein each lateral fenestration of the plurality of lateral fenestrations is circumferentially spaced apart.

15. An intraosseous access assembly, comprising:
a driver defined between a proximal driver end (PDE) and a distal driver end (DDE), the driver defining a gripping portion between the PDE and the DDE, the driver defining a plurality of prongs extending from the DDE;
a stylet extending from the DDE of the driver, the stylet extending between a distal stylet end (DSE) and a proximal stylet end (PSE);
a catheter hub removably couplable to the driver at the DDE, the catheter hub defining a plurality of apertures for receiving respective prongs of the plurality of prongs of the driver,
a catheter extending from the catheter hub, the catheter extending between a proximal catheter end (PCE) and a distal catheter end (DCE), the catheter defining a cutting edge and a longitudinal opening at the DCE,
wherein the stylet extending from the DDE of the driver is selectively received through the catheter hub and the catheter, the stylet comprising a tapered tip defined at the DSE and extending through the opening at the DCE, the cutting edge of the catheter and the tapered tip of the stylet form a cutting tip at the DSE.

16. The intraosseous access assembly of claim 15, wherein the cutting edge of the catheter comprises a pair of tapers defining a recessed portion at the longitudinal opening at the DCE.

17. The intraosseous access assembly of claim 15, wherein the gripping portion of the driver comprises an hourglass shaped portion between the PDE and the DDE.

18. The intraosseous access assembly of claim 15, wherein the catheter further defines a fenestration rearward from the longitudinal opening between the PCE and the DCE.

19. The intraosseous access assembly of claim 18, wherein the fenestration comprises a plurality of lateral fenestrations.

20. The intraosseous access assembly of claim 19, wherein each lateral fenestration of the plurality of lateral fenestrations is circumferentially spaced apart.
